(19) United States
(12) Patent Application Publication
(10) Pub. No.: US 2019/0096512 A1
(43) Pub. Date: Mar. 28, 2019

(54) COMPREHENSIVE VERIFICATION OF A PATIENT'S MEDICAL RECORD

(71) Applicant: KONINKLIJKE PHILIPS N.V., EINDHOVEN (NL)

(72) Inventor: Merlijn Sevenster, Haarlem (NL)

(73) Assignee: KONINKLIJKE PHILIPS N.V., EINDHOVEN (NL)

(21) Appl. No.: 15/772,573

(22) PCT Filed: Nov. 1, 2016
(86) PCT No.: PCT/EP2016/076267

§ 371 Date: May 1, 2018

Related U.S. Application Data
(60) Provisional application No. 62/251,123, filed on Nov. 5, 2015.

(51) Int. Cl. G16H 10/60; G06F 17/27; G06F 16/2455

(52) U.S. Cl. CPC G16H 10/60; G06F 16/24566; G06F 17/2765

(57) ABSTRACT

A rules verification system recursively resolves all conflicts that may be introduced by adding a finding to a set of findings of a patient. The system determines a set of findings that may be added to or removed from the existing set of findings such that the resultant set of findings do not violate any of the rules. Each rule is structured using an “IF {predicate}, THEN {consequent} ” construct, and if the findings provide a predicate that is true and a consequent that is false, the recursive resolution process includes, for each violated rule, finding a first set of findings that violate the predicate of the rule and a second set of findings that satisfy the consequent of the rule. The recursive process provides sets of branches that either end at a non-feasible solution, or end at a complete set of findings that satisfies all of the rules.

### FIELD OF THE INVENTION

This invention relates to the field of electronic medical records (EMR), and in particular to a system and method for identifying conflicts or contradictions in a patient's medical record and to provide one or more solution sets that each resolve all of these conflicts.

### BACKGROUND OF THE INVENTION

Over a lifetime, a person (hereinafter patient) may receive medical services from a variety of facilities and practitioners (hereinafter service providers). With the advent of computer technology, the patient's medical history may be maintained in an electronic form, as an electronic medical record (EMR). With the advent of computer networking, all of the patient's records from the variety of services providers may be integrated into a consolidated electronic medical record for this patient.

As time progresses, the patient's medical record grows, and, consequently, the likelihood of conflicts or contradictions appearing in the patient's record. Such conflicts may arise due to mistaken entries in the patient's records, unconfirmed findings, changes in the patient's characteristics, and so on. For example, if a patient begins a regime of weight loss and exercise, prior findings of high blood pressure, high cholesterol, diabetes, etc. may no longer be valid. A current provider of medical services to the patient may not be aware of the patient's prior conditions, and may not explicitly cancel such prior findings. In like manner, if an initial finding is determined to be incorrect, the service provider may not update the patient's record, particularly if the finding had no bearing on the eventually determined findings. Obviously, if the provider's entry was mistaken, the provider will be unaware of the mistake.

Retrospective research has shown that “nearly a quarter (24.1%) of finalized transthoracic reports and 10.1% of transesophageal reports had findings that were inconsistent with each other” (Chandra, Arling, Rock, & Spencer, 2010). Another evaluation (Spencer et al., 2015), which corroborated the earlier results, showed that 83% of reports violated at least one rule and that 62% of reports violated at least two rules.

Systems and methods have been developed to identify conflicts in findings within a patient's medical records, including U.S. patent application Ser. No. 13/999,831, “MEDICAL INFORMATION SYSTEM RULESET GENERATION AND/OR EVALUATION GRAPHICAL USER INTERFACE”, filed 31 May 2013 (and claiming priority to 3 Dec. 2010) for Robert Arling and Joseph Rock, which is incorporated by reference herein,. These systems are facilitated by the use of well defined “finding codes” for use among a variety of providers to update a patient's record. With well defined finding codes for particular medical findings, rules have been developed to facilitate “best practice” procedures, such as recommending missing findings that would facilitate more specific medical diagnoses. The use of these well defined finding codes also facilitate identifying conflicts among the findings in a patient's record.

As used herein, a “finding” for a patient includes any data element that may be associated with the patient. The person's individual data, such as age, gender, race, etc. are considered to be ‘findings’ related to the patient, as are the physiological data of the patient, such as blood pressure, blood sugar level, pulse rate, HDL, CDL, A1C, and so on. Any other data related to the patient may be considered a ‘finding’, including, for example, a diagnostic/finding of chronic depression, anxiety disorder, and so on. Treatments provided to the patient may also be considered ‘findings’, such as chemo-therapy, radiation-therapy, physical-therapy, prescribed medications, and so on.

In the aforementioned patent application, when a patient's record is updated and a conflict is found among the findings in a patient's record, the system identifies the conflict, and in some embodiments, the system provides a proposed solution to the conflict. As each solution is effected by the provider, the patient's record is updated and re-assessed to identify any remaining conflicts, and a potential solution may again be provided. This process continues until all conflicts are eliminated from the patient's record. As each new finding is entered into the patient's record, the record is reassessed to identify any conflicts introduced by this new finding, and another potential solution may be provided.

Although the example prior-art system, and others, may be very effective in eliminating, or at least reducing conflicts in a patient's record, by warning the user of a conflict each time the patient's record is updated, there are some situations that make using such systems frustrating, at best. A particularly frustrating situation is the occurrence of ‘cascading’ rule violations, wherein a conflict is first identified when the patient's record is updated, but the proposed solution introduces a different conflict. Solving the different conflict may again introduce another conflict, and so on. In some cases, this process of conflict1-solution1-conflict2-solution2-conflict3-solution3, and so on, may enter an infinite loop. When faced with cascading rule violations, the user must decide whether to terminate using the system to determine solutions, at which point the user must manually determine the cause(s) of these cascading rule violations.

FIG. 1 illustrates an example set of rules and findings that produce cascading rule violations. Four potential findings (A, X, Y, Z) are listed below:

A=Ejection fraction is severely reduced.

X=BMI<25 && ejection fraction>45%

Y=Ejection fraction is normal for body size

Z=Ejection fraction is normal.

For ease of reference herein, stating that a finding exists in a patient's record is equivalent to stating that the finding is true for this patient. If the finding is not found in the patient's record, this does not necessarily imply that the finding is false; it typically means that the finding has not yet been determined for this user.

Three rules 101 are included in this example, using the symbols A, X, Y, and Z for the above listed finding. In text form:
1. If the patient's BMI is less than 25 and the ejection fraction (the fraction of outbound blood pumped from the heart with each heartbeat) is found to be greater than 45% (finding X is true), then the patient's ejection fraction is normal for this body size (Y must be true).
2. If the patient's ejection fraction is normal for this body size (finding Y is true), then the patient's ejection faction is normal (finding Z must be true).
3. The patient's ejection fraction cannot be both: severely reduced (finding A is true) and normal (finding Z is true).

In this example, the patient's existing finding is A, at 110, and a new finding X is being added to this finding 115, producing a new set of existing findings {A, X}. The record verification system is invoked by this new entry, and reports that rule #1 (If X, then Y) is violated 130. This rule is violated because finding Y is not in the patient's records, but should be because finding X is in the patient's record. There are two possible solutions to this violation: either remove X from, or add Y to, the patient's record, as indicated in text box 130.
In this example, the user selects the “Add Y” option 135, producing a set of findings 140 of {A, X, Y}. These findings 140 violate the second rule because Z is not in the patient's record; the system reports this rule violation 150 to the user, along with potential solutions of either removing Y or adding Z. In this example, the user selects the “Add Z” option 155, producing a set of findings {A, X, Y, Z} 160.

The system then reports that this set of findings {A, X, Y, Z} 160 violate the third rule (A and Z are mutually exclusive), and offers the choice of either removing A or removing Z. If the user chooses 175 to remove A, the solution {X, Y, Z} does not violate any of the rules, and the patient's record will reflect these findings. However, if the user chooses 175 to remove Z, the system will continue to cascade through violated rules.

Although in this simple case the solution of removing A (i.e. having the practitioner determine that finding A is no longer true for the patient) would likely be apparent at 170, but in a more complex case, the solution may require a combination of additions and removals that are not immediately obvious.

It is significant to note that unless the cause of the cascading rule violations is corrected, the verification system will continually produce these cascading rule violations, particularly if a first step in the verification process is to verify that the existing patient records are conflict-free before determining whether a newly added finding introduces a conflict. In some verification systems, the user is provided the option of having the system ignore select conflicts, or to ignore select rules, to overcome the cascading rule violations, but such an option reduces the accuracy of the verification process, and may eventually result in a relatively ineffective verification.

### SUMMARY OF THE INVENTION

It would be advantageous to provide a rules verification method and system that does not lead a medical practitioner through a series of cascading rule violations. It would also be advantageous to provide a rules verification method and system that presents the medical practitioner with a complete set of changes that are required to be made to the patient's record to eliminate all conflicts.

To better address one or more of these concerns, in an embodiment of this invention, when a new finding is added to a patient's record, the rules verification system recursively resolves all conflicts that may be introduced by this added finding by determining a set of findings that may be added to or removed from the existing set of findings of the patient such that the resultant set of findings do not violate any of the rules. Each rule is structured using an “IF {predicate}, THEN {consequent}” construct, and if the findings provide a predicate that is true and a consequent that is false, the recursive resolution process includes finding a first set of findings that violate the predicate and a second set of findings that satisfy the consequent. The recursive process provides sets of branches that either end at a non-feasible solution, or end at a complete set of findings that satisfies all of the rules. The practitioner is provided with sets of changes that may be made to the patient's findings to produce each of these complete sets of findings that satisfy all of the rules, and may select and apply one of these sets of changes to the patient's findings.

In an example embodiment, the predicate and the consequent may include a quantifier that is applied to one or more of the plurality of findings, and each quantifier may be one of: all of the one or more plurality of findings; none of the one or more plurality of findings; at least one of the one or more plurality of findings; and At Most One of the one or more plurality of findings.

The system may be configured to provide every complete set of solutions that satisfy all of the rules; or, a termination criteria may be applied based on, for example, a time duration to provide the solutions, a predefined number of provided solutions, a measure of complexity of each potential solution, and so on.

### BRIEF DESCRIPTION OF THE DRAWINGS

The invention is explained in further detail, and by way of example, with reference to the accompanying drawings wherein:

FIG. 1 illustrates an example of cascading rule violations of a prior art rules verification system.

FIG. 2 illustrates an example flow diagram of an example rules verification system according to aspects of the current invention.

FIG. 3 illustrates an example flow diagram of a resolution process according to aspects of the current invention.

FIG. 4 illustrates an example flow diagram for determining a set of findings that violates the predicate of a rule.

FIG. 5 illustrates an example flow diagram for determining a set of findings that satisfy the consequent of a rule.

FIG. 6 illustrates an example determination of each complete set of findings that resolve the conflicts of the example of FIG. 1.

FIG. 7 illustrates an example block diagram of a rules verification system.

Throughout the drawings, the same reference numerals indicate similar or corresponding features or functions. The drawings are included for illustrative purposes and are not intended to limit the scope of the invention.

### DETAILED DESCRIPTION

In the following description, for purposes of explanation rather than limitation, specific details are set forth such as the particular architecture, interfaces, techniques, etc., in order to provide a thorough understanding of the concepts of the invention. However, it will be apparent to those skilled in the art that the present invention may be practiced in other embodiments, which depart from these specific details. In like manner, the text of this description is directed to the example embodiments as illustrated in the Figures, and is not intended to limit the claimed invention beyond the limits expressly included in the claims. For purposes of simplicity and clarity, detailed descriptions of well-known devices, circuits, and methods are omitted so as not to obscure the description of the present invention with unnecessary detail.

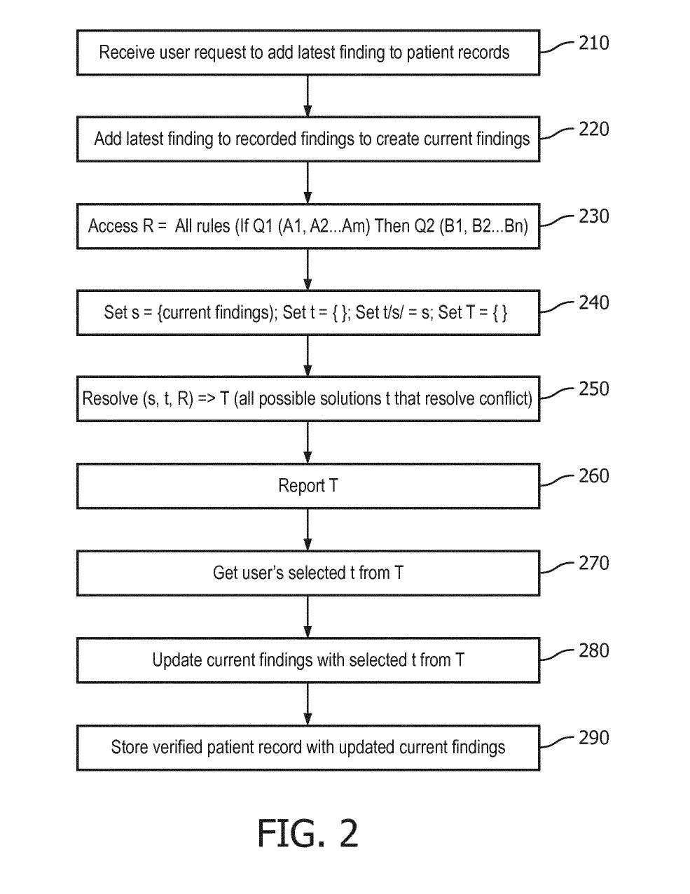

FIG. 2 illustrates an example flow diagram of an example rules verification system according to aspects of the current invention. This example system may be triggered by a user's (e.g. medical practitioner) addition of a new finding to a patient's record, at 210. The system adds the new finding to the patient's record, at 220, then accesses a set of rules that are to be applied to the updated set of findings, at 230. In this example embodiment, the rules are of the form:
IF Q1 (A1, A2, Am), THEN Q2 (B1, B2, Ban),
   Where Q1(A1, A2, . . . Am) is termed the ‘predicate’, and Q2(B1, B2, . . . Ban) is termed the ‘consequent’ of this IF . . . THEN . . . structure. The terms A1, A2, . . . B1, B2 . . . are the findings that are applicable to this rule. As noted above, as used herein the presence of a finding, such as A1, in an expression is a shorthand method of expressing “finding A1 is true for this patient”. Using the binary values of ‘0’ and ‘1’, where 0 represents ‘false’, and 1 represents ‘true’, an expression “A1->1” is also equivalent to “finding A1 is true for this patient”; and the expression “A1->0” is equivalent to “finding A1 is false for this patient”. It is significant to note that a finding may not appear in an expression, meaning that the finding may be true or false without affecting the expression. That is, the absence of “A1->1” in an expression does not imply that “A1->0”.

Q1 and Q2 are termed ‘quantifiers’, and express a relationship among the findings that must be true for the expressions Q1(A1, A2, . . . Am) and Q2(B1, B2, . . . Bn) to be true. In an example embodiment, the quantifiers Q1 and Q2 may be one of: All, None, At Least One, and At Most One. For example, if Q1 is “All”, then for All(A1 . . . Am) to be true, every one of the findings A1 . . . Am must be true. If Q1 is “At Most One”, then zero or one of the findings A1 . . . Am may be true, and all of the other findings must be false. In like manner, if Q1 is “None”, none of the findings A1 . . . Am may be true; and if Q1 is “At Least One”, one or more of the findings A1 . . . Am must be true.

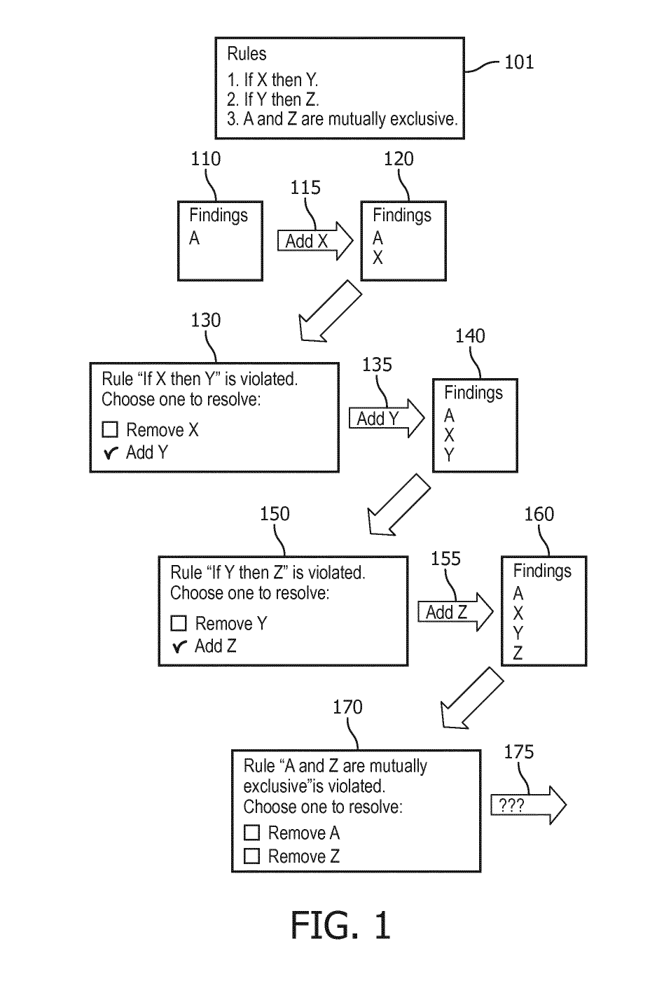

One of skill in the art will recognize that if the predicate or consequent has only one finding, either of the quantifiers “All” and “At Least One” will have the same effect. For example, the first rule of the example of FIG. 1 (“If X then Y”) may be expressed as “IF All{X} THEN All{Y}”, “IF All {X} THEN At Least One {Y}”, and so on.

In like manner, the third rule of the example of FIG. 1 (“A and Z are mutually exclusive”) may be expressed as “IF All{ } THEN At Most One {A, Z}”. Because the All{ } expression is independent of any particular finding, it is always true, and thus the meaning of this rule is “under all circumstances, only one of finding A or finding Z may be true”. This rule may also be expressed as two rules: “IF All{A->1} THEN All{Z->0}” and “IF All{Z->1} THEN All{A->0}”.

At 240, the rule verification system is initialized. In the example embodiment, ‘s’ represents the set of current findings (including the newly added finding), and ‘t’ represents findings that must be added to, or removed from, the current findings to eliminate (resolve) any and all conflicts that may exist in this set of current findings. The set ‘T’ includes all of the sets ‘t’ that resolve all of the conflicts. The set ‘t/s’ includes set ‘t’ and any of the findings in the current set ‘s’ that do not contradict the findings in the set ‘t’. Conceptually, the set ‘t/s’ includes all of the added or removed findings that are necessary to resolve all of the conflicts, while retaining all of the other existing findings that do not affect these necessary added or removed findings. That is, the set ‘t’ only removes findings from the current findings that are necessary to be removed to satisfy the given set of rules.

At 250, all of the sets of findings ‘t’ that resolve all conflicts in the current set of findings ‘s’ are collected in the set ‘T’, and this set ‘T’ is reported to the user at 260. Given each complete set of changes ‘t’ that can be made to the current findings for this patient to resolve any and all conflicts in the current set of findings ‘s’, the practitioner can assess each of these complete solutions to determine which solution to accept, at 270.

As noted above, a patient's condition over time may change, and the practitioner may easily be able to identify which proposed solution best matches the patient's current status. For example, if a prior weight of a patient provided a finding that the person was obese, a proposed solution to a current conflict may be to remove the finding that the patient was obese. If, in fact, the patient continues to be obese, the practitioner would not select this ‘solution’. If, on the other hand, the patient has lost weight, this prior ‘finding’ may no longer be valid, and the practitioner would concur with the solution of removing this finding from the current findings of the patient.

In some situations, additional tests or other information gathering procedure may need to be conducted to determine which proposed solution to accept, but the identification of which finding(s) is/are required to resolve the conflicts in the current findings will facilitate the identification of the tests/procedures required to select the appropriate solution set from among the plurality of solutions that resolve all conflicts in the current findings.

When the practitioner selects a given set of changes ‘t’ to the current findings, the system updates the patient's record to effect these changes ‘t’, thereby resolving all conflicts in the current findings in the patient's record.

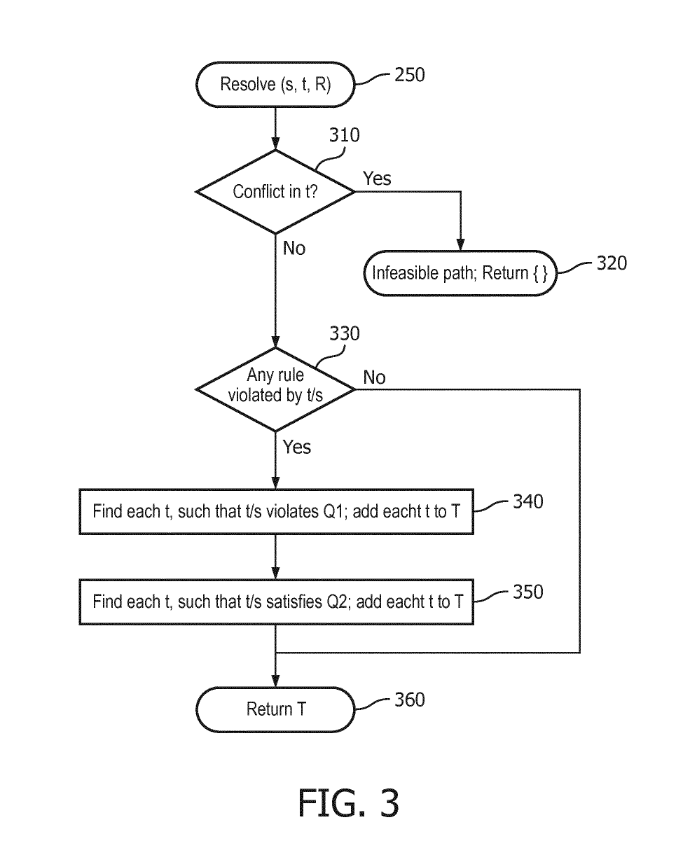

FIG. 3 illustrates an example flow diagram of a resolution process according to aspects of the current invention. As detailed further below, this resolution process is a recursive process, such that each possible resolution of a rule violation include a determination of whether this resolution includes a violation of another rule; and, if so, each of the possible resolutions of the violation of this other rule are included in the possible resolutions of the original rule violation.

As noted above, the set‘t’ of changes to the current findings ‘s’ are the required changes to resolve the conflict(s) introduced by the current findings. The findings in the set ‘t’ may conflict, and often do, with the current findings ‘s’ (so as to identify the removal of conflicting findings in s), but they may not conflict with prior required findings in ‘t’. When, at 310, such a conflict situation arises, the thus far developed changes to the set ‘t’ will be impossible to implement; accordingly the system identifies this particular set ‘t’ of changes as infeasible, and return a null set, at 320.

If, on the other hand, at 310, the thus far developed required changes in the set ‘t’ do not contain a conflict, the system determines, at 330, whether all of the conflicts in the current set of findings ‘s’ have been resolved by the changes identified in set ‘t’. If so, resolution of all conflicts is complete, and the process returns, at 360. This thus far developed set ‘t’ of this solution will have been included in the set T of complete sets of solutions ‘t’, as detailed further below.

If, at 330, the system determines that a rule is violated by the thus far developed set ‘t/s’, the system resolves this current rule violation by determining each set ‘t’ of changes that produce a set ‘t/s’ that violates the predicate of this current rule, at 340. A current rule violation occurs when the predicate of the rule is true and the consequent of the rule is false. By changing the findings, via a set ‘t’, such that the predicate of the rule is false, the rule is not invoked, and no violation occurs.

At 350, each set ‘t’ of changes to the findings that provides a set of findings ‘t/s’ that satisfies the consequent of the current rule is determined. As noted above, a current rule violation occurs when the predicate of the rule is true and the consequent of the rule is false. By changing the findings via a set of changes ‘t’, such that the set of findings ‘t/s’ satisfy the consequent of the current rule, the rule is satisfied, and no violation occurs.

Each set ‘t’ that is determined to violate the predicate of the currently violated rule, or satisfy the consequent of the currently violated rule is returned to the ‘next higher up’ rule resolution, until eventually the sets ‘t’ that either violate the predicate of the first identified rule violation of the current set of finding ‘s’, or satisfy the consequent of this first identified rule violation, are returned and added to the set ‘T’ of solutions that eliminate all of the conflicts that exist in the current set of findings ‘s’.

Effectively, the recursive process of FIG. 3 creates a tree comprising a plurality of branches that either violate the predicate of a rule that is violated by the current set of findings ‘s’, or satisfy the consequent of this violated rule. The terminal leaves of the branches are either a null set, indicating that this particular branch is not feasible, or a terminal leaf at the end of a branch that either violates the predicate, or satisfies the consequent of the rules that are violated by the current set of findings ‘s’. Because the ‘final’ resolution at the end of each feasible branch finds a solution that satisfies all of the conflicts in the set of findings ‘t/s’, the implementation of the set of changes ‘t’ is assured to resolve all of the conflicts in the original set of findings ‘s’. Accordingly, the choice of the first identified rule violation in the original set ‘s’ is immaterial to the resultant set ‘T’ of all solutions ‘t’.

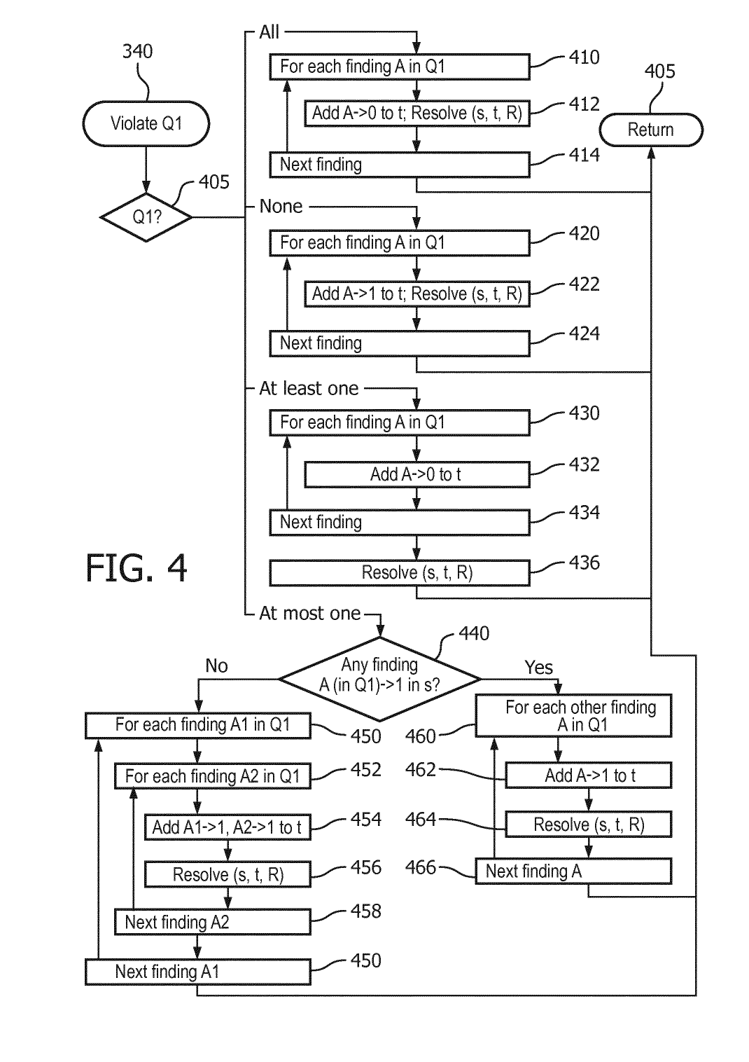

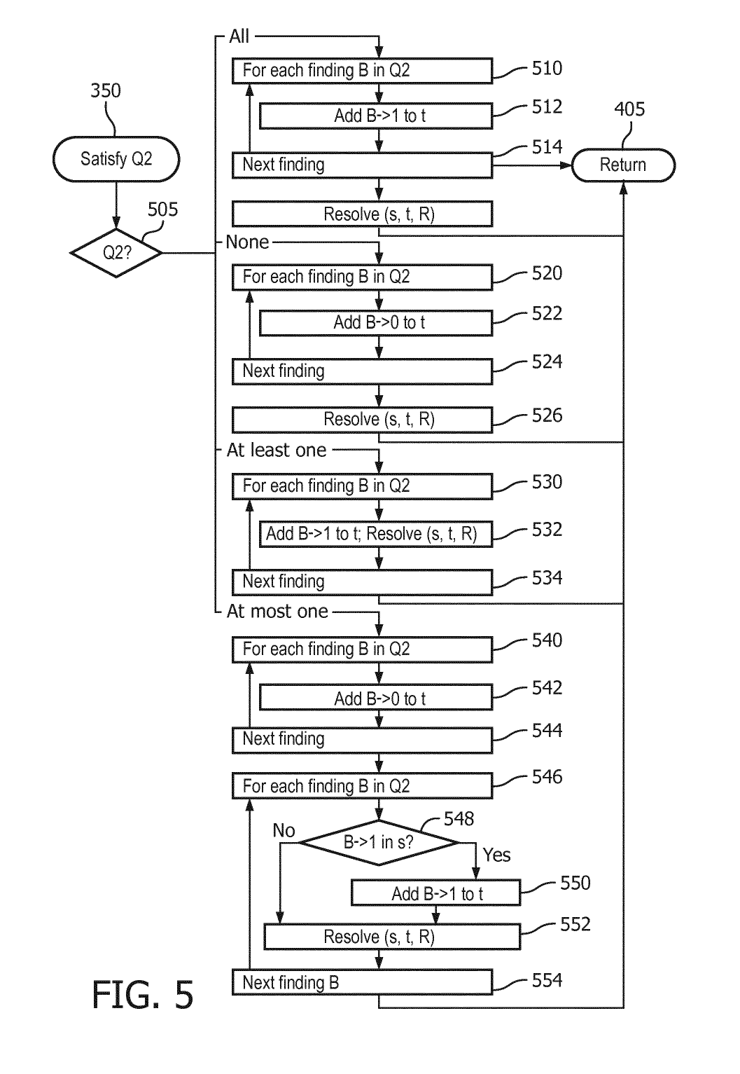

FIGS. 4 and 5 illustrate more detailed flow diagrams that may be used to implement the recursive processes of determining the set of changes ‘t’ necessary for ‘t/s’ to violate the predicate of the currently violated rule (340 of FIG. 3), and determining the set of changes ‘t’ necessary for ‘t/s’ to satisfy the consequent of the currently violated rule (350 of FIG. 3).

FIG. 4 illustrates an example flow diagram for determining 340 a set of findings that violates the predicate of a rule, assuming that the predicate of the rule is of the form Q1 {A1, A2 . . . Am}, wherein Q1 is one of “All”, “None”, “At Least One”, and “At Most One”. At 405, Q1 is identified as being one of these quantifiers.

If Q1 is “All” then the predicate will be violated if at least one of the findings A1 . . . Am is false (->0). The loop 410-414 steps through each A of the findings A1 . . . Am, and adds the finding that A is false (A->0) to the set of changes ‘t’ that violates the “All {A1, A2 . . . Am}” predicate. Conflicts in the new set of findings ‘t/s’ produced by the added finding of A->0 are resolved, at 412, using the process illustrated in FIG. 3 (hence the term ‘recursive’ resolution).

If the existing set of required changes ‘t’ includes setting the finding to be true (A->1), this addition of (A->0) to the set ‘t’ is contradictory, and this set of changes ‘t’ are determined to be infeasible (320 in FIG. 3).

If, on the other hand, the current set of changes ‘t’ do not specify that this finding A is true, this addition (A->0) to the findings ‘t’ is accepted. It is significant to note that the current findings ‘s’ may include a contradictory finding that A is true, and accepting this opposing finding is equivalent to requiring that the practitioner determines that this prior finding can be reversed. If the current findings ‘s’ do not include a finding that contradicts this addition (A->0), the practitioner need only concur that finding A is false (without contradicting a prior finding). In some situations, the practitioner may make this determination based on the practitioner's assessment of the patient's record; in other situations, the practitioner may require that additional tests or other information gathering processes be conducted to verify the proposed added finding.

This determination of an addition to the set of changes ‘t’ corresponding to setting each of the findings A1 . . . Am to false, results in ‘m’ sets of changes ‘t’. Of these ‘m’ sets of changes ‘t’, some or all may be infeasible (i.e. conflict within ‘t’); and each of the remaining feasible set of changes ‘t’ are returned as changes to ‘t/s’ that will resolve the conflict in the currently violated rule produced by the current set of findings ‘s’. That is, the loop 410-414 will produce 0 to m sets of changes ‘t’ (branches off the branch ‘t’ before adding A->0) that each provide a set ‘t/s’ that violates the predicate of the current rule.

If Q1 is “None”, then the predicate will be violated if at least one of the findings is true. The loop 420-424 is similar to the loop 410-414, except that each finding A is set to true (A->1) in 422, as contrast to setting finding A to false (A->0) in 412. As in the loop 410-414, m sets of changes ‘t’ will be produced, some of which may be infeasible, the remainder being 0 to m sets of changes ‘t’ that each provide a set ‘t/s’ that violates the predicate “None(A1, A2 . . . Am)” of the currently rule that is violated by the set of findings ‘s’, thereby eliminating this violation of the current rule.

If Q1 is “At Least One”, then the predicate will be violated only if all of the findings A1 . . . Am are false. That is, as contrast to the aforementioned ‘All’ and ‘None’ quantifiers that produce m sets of changes ‘t’ to violate the predicate, only one solution (all findings are false) will violate the “At Least One (A1, A2 . . . Am)” predicate. Accordingly, the loop 430-434 sets all of the findings to false (A->0), at 432, and conflicts introduced by the addition of this single set of findings in ‘t’ to violate the predicate of the current rule are resolved, at 436.

If Q1 is “At Most One”, then the predicate “At Most One (A1, A2 . . . Am)” will be violated if at least two of the findings A are true. When this process is invoked (at 340 in FIG. 3), the existing predicate must be true (otherwise, the rule cannot be violated); therefore either none of the findings A in the current ‘t/s’ are true, or one of the findings A in ‘t/s’ is true. (If two or more of the findings A are true in ‘t/s’, this rule is not violated, because the “At Most One” predicate is violated.

At 440, the set ‘t/s’ is assessed to determine whether 0 or 1 of the findings A are true. If one of the findings A in ‘t/s’ is true, the loop 460-466 adds the finding that each of the other findings A1 is true, at 462. As each finding A1->1 is added to the set of changes ‘t’, any conflicts remaining in the set of findings ‘t/s’ are resolved, at 464. Thus, m-1 sets of findings are defined, some of which will be infeasible (conflict in ‘t’); accordingly, 0 to m-1 feasible sets of changes ‘t’ that produce a set of findings ‘t/s’ that violate the predicate of the current rule will be produced.

If, at 440, none of the findings A in the set of findings ‘t/s’ are currently set to true, the nested loops 452-458 within 450-460 produce sets of changes ‘t’ that include two settings A1, A2 that are set to true, at 454, thereby violating the “At Most One (A1, A2 . . . Am)” predicate of the current rule. Any conflicts remaining in the set of findings ‘t/s’ provided by these additions are resolved, at 456. Up to m*(m-1)/2 sets of ‘t’ will be produced, some of which will be infeasible (conflict in ‘t’); accordingly 0 to m*(m-1)/2 sets of changes ‘t’ that each provides a set ‘t/s’ that resolves all of the conflicts in the current rule are determined.

FIG. 5 illustrates an example flow diagram for determining 350 a set of findings that satisfies the consequent of a rule, assuming that the predicate of the rule is of the form Q2 {B1, B2 . . . Bn}, wherein Q2 is one of “All”, “None”, “At Least One”, and “At Most One”. At 505, Q2 is identified as being one of these quantifiers.

If Q2 is “All” then the consequent will be satisfied only if all of the findings B1 . . . Bn are true (->1). The loop 510-514 steps through each B of the findings B1 . . . Bn, and adds the finding that B is true (B->1) to the set of changes ‘t’ that satisfy the “All {B1, B2 . . . Bn}” predicate, at 512. Conflicts in the new set of findings ‘t/s’ that are remaining after the added finding that all findings B1 . . . Bn are true are resolved, at 516, using the process illustrated in FIG. 3.

If the existing set of required changes ‘t’ includes setting any of the findings to be false (B->0), the addition of (B->1) to the set ‘t’ is contradictory, and this set of changes ‘t’ are determined to be infeasible (320 in FIG. 3).

If, on the other hand, the current set of changes ‘t’ do not specify that any of the findings B are false, the setting of all of the findings B1 . . . Bn to be true in ‘t’ is accepted. It is significant to note that the current findings ‘s’ may include a contradictory finding that B is false, and accepting this opposing finding is equivalent to requiring that the practitioner determines that this prior finding can be reversed. If the current findings ‘s’ do not include a finding that contradicts these additions (all B->1), the practitioner need only concur that all of the findings B1 . . . Bn are true (without contradicting a prior finding). As noted above, the practitioner may require additional tests, or other information, to confirm that this addition is proper.

This determination of an addition to the set of changes ‘t’ corresponding to setting all of the findings B1 . . . Bn to true, results one set of changes ‘t’, which may be infeasible (i.e. conflict within ‘t’). If the set of changes ‘t’ are determined to be feasible, it is returned as a set of changes that produce a set ‘t/s’ that will resolve the conflict in the currently violated rule produced by the current set of findings ‘s’.

If Q2 is “None”, then the consequent will be satisfied if none of the findings in set ‘t/s’ is true. The loop 520-524 is similar to the loop 510-514, except that each finding B is set to false (B->0) in 522, as contrast to setting each finding B to true (B->1) in 512. As in the loop 510-514, one set of changes ‘t’ will be produced, which may be infeasible. If the set of changes ‘t’ is feasible, set ‘t’ is returned as the set of changes required to satisfy the consequent “None(B1, B2 . . . Bn)” of the currently rule.

If Q2 is “At Least One”, then the consequent will be satisfied if at least one of the findings B1 . . . Bn is true. As noted above, this process would not be invoked if the consequent of the rule were true; that is, all of the findings B1 . . . Bn must either be absent in the patient's record, or set to false in the patient's record. Accordingly, the loop 530-534 sets each of the findings B to true (B->1) in ‘t’, and any conflicts remaining in the resultant set ‘t/s’ are resolved, at 532. In this loop 530-534, n sets of changes ‘t’ are introduced, some of which may be infeasible. Accordingly 0 to n sets of feasible changes ‘t’ are provided, each of these sets of changes ‘t’ providing a set of findings ‘t/s’ that resolve all of the conflicts in the current set of findings ‘s’ with regard to the current rule.

If Q2 is “At Most One”, then the consequent “At Most One (B1, B2 . . . Bn)” will be satisfied if either zero or one of the findings is true. The current findings ‘t/s’ must include at least two of the findings being true to have invoked this rule violation process. In the loop 540-544, all of the findings B1 . . . Bn are set to false (B->0) in ‘t’, at 542, overriding all of the findings in set ‘s’ that indicate that B is true (B->1). Thereafter, in the loop 546-554, only the findings of B that are set to be true in the original set ‘s’, at 548, need to be assessed to determine the effects of having this finding B being the only finding among B1 . . . Bn that is true. At 550, this finding B is set to true (B->1) in t, and any conflicts with the resultant set ‘t/s’ are resolved, at 552.

In FIGS. 4 and 5, each of the feasible sets of changes ‘t’ that provides a set of findings ‘t/s’ that resolve all conflicts among the findings based on the current rule are returned to the process that invoked the current resolution process. Accordingly, at the uppermost level, when a conflict is identified in the current set of findings ‘s’, each of the returned sets of changes ‘t’ are assured to resolve all conflicts that may be present in the current set of findings ‘s’.

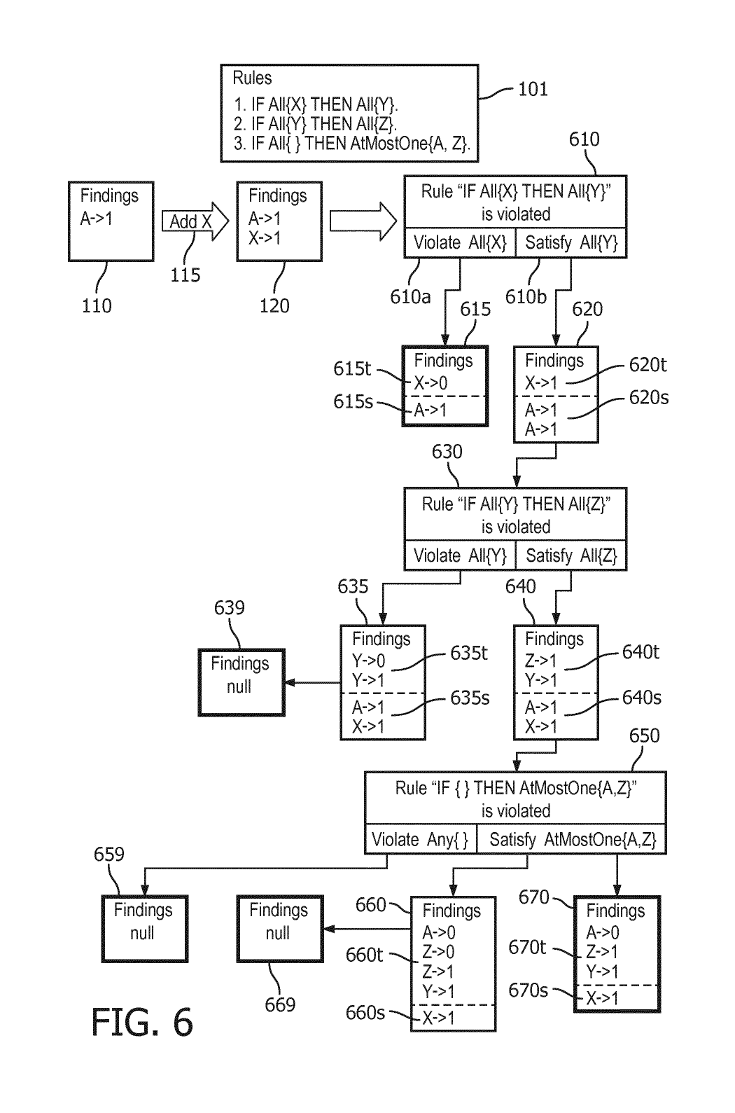

FIG. 6 illustrates an example determination of each complete set of findings that resolve the conflicts of the example of FIG. 1, using the example process of FIG. 2. The service provider adds finding X 115 to the patient's current findings {A} 110, to produce a set of findings {A, X} 120 that are assessed by the conflict resolution process (250 of FIG. 2). These findings 120 are assessed with respect to each of the rules 101 of FIG. 1, which are cast in the form “IF Q1(A1, A2 . . . Am) THEN Q2(B1, B2 . . . Bn)” rules 601.

As in the example of FIG. 1, a user (e.g. service provider) desires to add finding X 115 to a current set of patient findings A 110. The resultant set 120 correspondingly includes findings {A,X}, hereinafter refered to as the set of findings ‘s’. The rule verification system processes this new set ‘s’ 120 to verify that none of the rules 601 are violated, or, if any rule 610 is violated, to identify solutions that remove all violations in the set 120.

The finding X violates the first rule “IF All{X} THEN All{Y}”, at 610, because finding Y is not included in the set 120. There are two potential resolutions of this conflict: violate All{X} 610a, or satisfy All {Y} 610b.

Referring to FIG. 4, to violate the finding All{X}, at least one of the findings must be set to false. There is only one finding (X) in the ‘All’ quantifier, and the loop 410-414 introduces the finding (X->0) to the set of findings ‘t’ 615t. The resultant set ‘t/s’ 615 includes this set of findings ‘t’ 615t (above the dashed line) plus all findings is ‘s’ that do not conflict with the set of findings in ‘t’, 615s (below the dashed line).

The set of findings ‘s’ is {A, X}, and X being in this set (i.e. X->1) conflicts with the finding (X->0) that would need to be added to the solution set ‘t/s’ to resolve the current conflict. Accordingly, only the finding A, which does not conflict with the findings in ‘t’ (X->0), is included in the resultant set ‘t/s’. This resultant set ‘t/s’ includes {A->1, X->0}, which does not violate any of the rules 601. Accordingly, this solution resolves the conflicts introduced by adding finding X 115 to the set of findings {A} 110.

Effectively, this solution prevents the user from adding finding X 115 to the set of findings {A} 110, which may be the appropriate solution if, for example, entering the finding X was the result of a typographic mistake. Accordingly, the rule verification system of this invention will often avoid the entry of erroneous findings by notifying the user that the proposed finding is in conflict with existing findings.

In the alternative, the conflict in the set ‘s’ {A, X} can be resolved by satisfying the consequent of the currently violated rule “IF All{X} THEN All{Y}”. As indicated in steps 510-516 of FIG. 5, the satisfaction of All{Y} requires setting all of findings (in this case, the single finding Y) to true (Y->1). The resultant set of findings ‘t/s’ 620 includes this additional finding (Y->1) 620t in ‘t’ plus the findings in ‘s’ {A, X} that do not conflict with the findings in ‘t’ {(Y->1)} 620t. Neither finding A nor finding X conflict with the finding Y->1; accordingly, findings A and X are included 620s in the solution set ‘t/s’ of {Y->1; A->1; X->1} 620.

The solution set ‘t/s’ of {Y->1; A->1; X->1} 620 is assessed to determine whether any rules are violated, and the second rule “IF All{Y} THEN All{Z} is found to be violated 630. As in the example of the first rule being violated 610, the two potential solutions are to violate All{Y} (Y->0) or satisfy All {Z} (Z->1).

The violation of All {Y} requires adding Y->0 to the current set of added findings ‘t’ (Y->1) 620t. However, requiring Y->0 and Y->1 in the set of changes ‘t’ will be impossible to implement; accordingly, this branch of the potential solution sets is infeasible, and a ‘null’ response is returned 639 to indicate this fact.

The satisfaction of All{Z} requires adding Z->1 to the set of changes ‘t’ 620t. This finding does not conflict with the current set of changes ‘t’ 620t, and thus Z->1 is added to the set of changes ‘t’ 640t. Neither of the findings in ‘s’ (A->1; X->1) conflict with this finding, so these set of findings are included in the resultant set of findings t/s {A->1; X->1; Y->1; Z->1} 640.

The solution set ‘t/s’ {A->1; X->1; Y->1; Z->1} 640 is assessed to determine whether any of the rules 601 are violated, and the third rule “IF Any{ } THEN AtMostOne {A, Z}” is found to be violated because both A->1 and Z->1 are included in this solution set ‘t/s’ 640.

To resolve this conflict, either the predicate Any{ } must be violated, or the consequent AtMostOne {A, Z} must be satisfied. The predicate Any{ } does not include a finding, and thus a change of finding that violates this predicate does not exist. Accordingly, a solution along this branch of potential solutions is not feasible, and a null response is returned 659.

To satisfy the consequent AtMostOne{A,Z}, the process of steps 540-554 of FIG. 5 is applied to the currently viable solution set ‘t/s’ 640. In the loop 540-544, each of the findings in the consequent is ‘temporarily’ set to false (A->0, Z->0) in the set of changes {t}. This setting is temporary because the check for whether a subsequently added finding conflicts the set ‘t’ is not performed until the findings are resolved (310 in FIG. 3); until then, a subsequently added finding merely replaces the ‘temporary’ finding.

The loop 546-554 is performed for each of the findings A and Z. At 548, the finding A is compared to the elements of the set of original findings ‘s’ {A, X}, and is found to be in ‘s’ (“yes”). The set ‘t’, which includes A->0 and Z->0 in ‘t’ {Z->0; Y->1; Z->1} and the resultant set ‘t/s’ (A->0, Z=>0, Z->1, Y->1; X->1} 660 is then resolved 552. Because the set ‘t’ 660t includes a contradiction, {Z->0; Z->1}, this branch of the potential solutions is infeasible, and a null set is returned, at 669.

It is significant to note that because the process of this invention facilitates the identification and elimination of conflicting changes, the aforementioned possibilities of infinitely cascading solutions in the prior art are avoided by preventing solutions that first suggest setting a finding to be true, then subsequently setting this finding to false, and vice versa.

The loop 546-554 is then applied for the finding Z. At 548, Z is not found in ‘s’ and Z->1 is added to ‘t’, replacing the (termporary) prior assignment Z->0 at step 542. The set of changes ‘t’ still includes A->0, from step 542, and the resultant set ‘t’ is {A->0, Z->1, Y->1} 670t (the ‘addition’ of Z->1 is redundant, because Z->1 is already in set ‘t’ 640t). The resultant set of findings ‘t/s’ 670 includes these findings in ‘t’ {A->0, Y->1, Z->1} 670t plus the findings in ‘s’ {A->1, X->1} that do not conflict with these findings in ‘t’. Since the finding A->1 in ‘s’ conflicts with A->0 in ‘t’, this finding A->1 is not included in the resultant set ‘t/s’ {A-0, Z->1, Y->1; X->1) 670.

An assessment of these findings ‘t/s’ 670 does not reveal a violated rule 601, and the set of findings ‘t’ 670t is returned as a set of changes that may be used to resolve the conflicts introduced by the addition 115 of finding X to the original set of findings {A} 110. This set of changes {A-0, Z->1, Y->1} requires the reversal of the finding A->1 that was in the original set of findings {A} 110. As noted above, such a reversal may be warranted when the patient's condition has changed (due to medications, exercise, weight loss, etc.). Accordingly, the rule verification system of this invention can be seen to facilitate the removal of outdated findings, or previously mistaken entries of findings.

After finding all of the solutions that either violate the predicate or satisfy the consequent of the first determined violated rule (IF All{X} THEN All{Y}), the rule verification system returns all ‘T’ of the possible sets of changes ‘t’ that will each resolve the conflicts introduced by the addition of finding X to the original set of findings {A} to form a set of findings {A, X}. In this example, the returned sets of changes ‘t’ include:
1. {X->0} 615t; and
2. {A->0; Y->1; Z->1} 670t.

In this example, the user is provided two options for resolving the conflict in {A, X}, each of which requires reversal of one of the findings A, X. Such a reversal of a prior finding A, or a new finding X, would be counter-intuitive, and in a conventional step-by-step resolution of conflicts, the rule violations will continue to cascade until the reality that either A or X must be fault is recognized by the user. Using the current invention in this example situation, the user is immediately presented with the reality that either A or X must be false, because no other solutions are feasible, and cyclic resolution attempts are avoided.

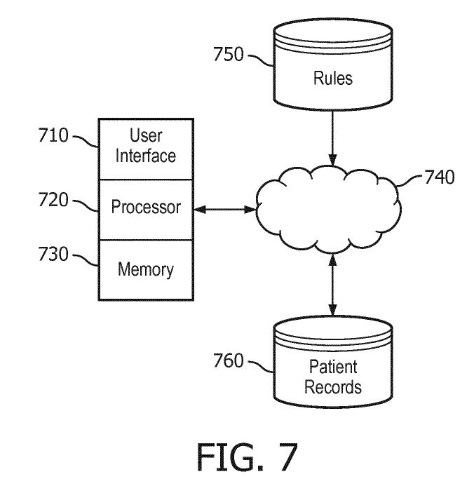

FIG. 7 illustrates an example block diagram of a rules verification system of this invention. The processor 720 is configured to execute one or more programs that provide the rules verification process of this invention, as detailed above. The processor 720 receives, via a user interface 710, an identification of a patient and a rule set 750 that is to be applied to any changes to the findings in this patient's records 760. (Although the invention has been presented in the context of the addition of a new finding, this new finding could be that a prior finding is no longer valid (i.e. a ‘deletion’, rather than an ‘addition’ per se)). Communications among the elements of the rules verification system may be via a network 740.

The processor may copy the particular patient's findings to a memory 730, to facilitate efficient access to each finding, and may also copy some or all of the rules that are to be applied to these findings to the memory 730. In some embodiments, particularly when the rule set 750 is large, rules may be added (cached) to the memory as they are encountered during the processing of the patient's findings. That is, each rule may include an identification of all of the findings that are applicable to the rule; as each finding of the patient is accessed, each of the rules that relate to the finding is loaded into the memory 730, if it is not already in the memory 730.

When the user identifies a change to be made to the findings in the patient's record, the processor 720 applies the applicable rules 750 to identify any conflicts within the resultant current set of findings. If a conflict is found, the processor may apply the example procedures detailed above in FIGS. 2-5 to identify one or more sets of changes that would be necessary to eliminate all conflicts in the patient's current set of findings.

Each of the determined sets of changes that would eliminate all conflicts in the patient's set of findings is presented to the user via the user interface 710. After assessing the sets of changes, the user selects a given set of changes, and the processor 720 implements these changes to the patient's record 760. As noted above, the selection of a given set of changes may not be immediate, because the user may need to collect additional information before some or all of the changes are determined to be acceptable to the user.

While the invention has been illustrated and described in detail in the drawings and foregoing description, such illustration and description are to be considered illustrative or exemplary and not restrictive; the invention is not limited to the disclosed embodiments.

For example, it is possible to operate the invention in an embodiment wherein the set of solutions T may not include all of the possible feasible solutions. As can be well imagined, with a large set of rules being applied to a complex patient record, the number of feasible solutions to conflicts may be extremely large as the combinations and permutations of each solution to a rule grows. To limit the number of solutions presented to the user, a ‘complexity’ level may be predefined such that if the set ‘t’ at any branch of an as-yet feasible solution exceeds changing a predefined number n of changes in findings, the solutions provided by subsequent branches in the solution are defined to be ‘too complex’ and further processing along this branch is terminated. The solution set ‘T’ will therefore include “all feasible solutions below complexity level n”. If the user decides that the reported solutions below this complexity level are not acceptable, the complexity level may be increased and the process repeated (or resumed).

Other termination criteria may be used to limit the number of solutions presented to the user, including, for example, terminating the verification process after a given elapsed time. In another example embodiment, the aforementioned complexity level may not be based on the number of changes in ‘t’, but based on a composite likelihood that the proposed changes may be acceptable. For example, the likelihood of a finding that the patient has an elevated temperature is likely to be greater than the likelihood of a finding that the patient has some rare disease, and solution branches that include a finding of an elevated temperature may be less likely to be terminated than branches that include a finding of the rare disease. Similarly, if branches must be terminated prematurely, the termination criteria may be based on an assigned ‘cost’ of determining the finding. If a branch includes a finding that requires measuring the patient's temperature, that branch may be less likely to be terminated than one that requires an invasive procedure, such as a biopsy, or a costly procedure, such as an MRI. As each branch is taken during the verification process, a cost or complexity measure may be accumulated for each finding required by that branch thus far. When the measure exceeds a given threshold, the further processing beyond that branch may be terminated.

In like manner, each solution ‘t’ may be displayed as soon as it is determined, and the user may be provided the option of changing the findings and restarting or resuming the verification process. For example, if a large portion of the displayed solutions includes adding a finding that the user knows to be false, the system may be configured to allow the user to prevent the system from pursuing solutions along such paths.

For example, certain ‘findings’ such as gender, age, and so on, may be identified as ‘persistent’, and the system may be prevented from initiating any branch that conflicts with such persistent findings. For example, if these findings are included in a set ‘p’, the decision block 310 of FIG. 3 may be restated as “Conflict in t, or p?” In the above example of enabling a user to prevent the system from following paths that are known to be erroneous, the user may be allowed to temporarily define a particular finding to be ‘persistent’ for the duration of the current rule verification process.

Also, although the invention is presented in the context of validating a newly entered finding, one of skill in the art will recognize that the invention may be applied to an existing patient's record, wherein any one of the existing findings in the patient's record may be considered to be the ‘new’ finding that has been added to all of the other existing findings. In an example embodiment, a facility may initiate the verification process of this invention for a given patient before the patient arrives for the gathering of new findings, so that any conflicts in the patient's record may be resolved before the potential introduction of additional conflicts caused by the newly gathered findings.

Similarly, an embodiment of this invention may be used to ‘merge’ records related to the patient at two (or more) provider databases. The “new finding” as used herein is not limited to a single finding, and may include a new set of findings. That is, the merging of two records may be considered to be the addition of a new set of findings (from one database) to an existing set of findings (from the other database).

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

1. A non-transitory computer-readable medium that includes a program that, when executed by a processor, causes the processor to:
receive a plurality of existing findings for a patient;
receive a new finding for the patient;
append the new finding to the plurality of existing findings;
access a medical rules database that includes a plurality of medical rules;
apply the plurality of rules to the plurality of findings to identify at least one conflict among the findings based on the rules;
upon identifying at least one conflict among the findings based on the rules:
recursively resolve each conflict to determine a plurality of complete sets of solutions that each resolve all of the conflicts to satisfy all of the rules, and
present each of the plurality of complete sets of solutions to a user,
wherein:
each medical rule being of the form: IF {predicate} THEN {consequent}; and
recursively resolving each conflict of a rule, includes determining:
a first set of solutions that do not satisfy the predicate of the rule, and
a second set of solutions that satisfy the consequent.

2. The medium of claim 1 wherein:
each set of solutions includes a set of findings that add to or subtract from the plurality of existing findings for the patient.

3. The medium of claim 1, wherein
at least one of the predicate and the consequent includes a quantifier that is applied to one or more of the plurality of findings.

4. The medium of claim 3, wherein each quantifier includes one of:
all of the one or more plurality of findings;
none of the one or more plurality of findings;
at least one of the one or more plurality of findings; and
At Most One of the one or more plurality of findings.

5. The medium of claim 1, wherein the program causes the processor to provide every complete set of solutions that satisfy all of the rules.

6. The medium of claim 1, wherein the program causes the processor to provide the plurality of sets of solutions based on a termination criteria.

7. The medium of claim 6, wherein the termination criteria includes at least one of a time duration to provide the solutions and a predefined number of provided solutions.

8. The medium of claim 6, wherein the termination criteria includes a measure of complexity associated with each potential solution.

9. The medium of claim 6, wherein the termination criteria includes a measure of cost associated with each potential solution.

10. The medium of claim 6, wherein the termination criteria includes a measure of a likelihood that each added finding in each proposed solution may be true.

11. A rules verification system comprising:
a processor;
a user interface coupled to the processor; and
a memory coupled to the processor;
wherein the processor is configured to:
access a patient's record of current findings, based on an identification of the patient via the user interface;
access a plurality of rules associated with the existing findings, each rule being in to form of “IF predicate THEN consequent”;
identify a violation of at least one rule of the plurality of rules caused by the current findings;
resolve the violation of the rule to identify at least one set of changes to the current findings that will eliminate all rule violations in the current findings;
receive, via the user interface, a user's selection of a select set of changes from the at least one set of changes;
apply the select set of changes to the current findings; and
update the patient's record of current findings based on the application of the select set of changes.

12. The system of claim 11, wherein the processor resolves the violation of the rule by identifying at least a first set of changes, if any, that will violate the predicate of the rule, and at least a second set of changes, if any, that will satisfy the consequent of the rule.

13. The system of claim 11, wherein the processor resolves the violation of the rule by recursively resolving violations of each rule that are introduced when the processor identifies each change of the set of changes that is required to resolve the violation.

14. The system of claim 11, wherein the processor terminates the identification of the at least one set of changes that resolve the violation of the rule based on a termination criteria that includes at least one of: a complexity measure of the at least one set of changes, a cost measure of the at least one set of changes, and a time duration to determine the at least on set of changes.

15. A method comprising:
receiving a plurality of existing findings for a patient;
receiving a new finding for the patient;
appending the new finding to the plurality of existing findings;
accessing a medical rules database that includes a plurality of medical rules;
applying the plurality of rules to the plurality of findings to identify at least one conflict among the findings based on the rules;
upon identifying at least one conflict among the findings based on the rules:
recursively resolving each conflict to determine a plurality of complete sets of solutions that each resolve all of the conflicts to satisfy all of the rules,
presenting each of the plurality of complete sets of solutions to a user,
receiving a selection of a select set of solutions by the user, and
correcting the existing findings of the patient by implementing the select set of solutions
wherein:
each medical rule being of the form: IF {predicate} THEN {consequent}; and
recursively resolving each conflict of a rule, includes determining:
a first set of solutions that do not satisfy the predicate of the rule, and
a second set of solutions that satisfy the consequent.
